(19) United States
(12) Patent Application Publication
(10) Pub. No.: US 2026/0232300 A1
(43) Pub. Date: Aug. 13, 2026

(54) APPARATUS, KIT, SYSTEM AND METHOD FOR CAPTURING A URINE SAMPLE FOR TESTING

(71) Applicant: Ryan W. DODD, Wolfeboro, NH (US)

(72) Inventor: Ryan W. DODD, Wolfeboro, NH (US)

(21) Appl. No.: 19/047,914

(22) Filed: Feb. 7, 2025

(51) Int. Cl. A61B 10/00

(52) U.S. Cl. CPC A61B 10/007

(57) ABSTRACT

The present invention is directed to an apparatus, kit, system and method for capturing a urine sample from a patient who is using an external catheter system, without removing urine from the collection canister of the external catheter system. The collection apparatus is connected in-line between the urine collection device and collection canister of an external catheter system to conveniently capture real-time urine samples. Upon collecting the sample, the urine collection device can be disconnected and the external catheter system can continue with normal operation.

### BACKGROUND OF THE INVENTION

### 1. Field of the Invention

The present invention is generally directed to urine sample collection, and is more specifically directed to an apparatus, kit, system and method for obtaining a urine specimen or sample for testing from a patient who is utilizing an external catheter system, without the need to remove urine from the collection canister of the external catheter system.

### 2. Background

External catheter systems are increasingly being used to collect urine from patients who are bedridden or who have limited mobility in hospitals and other settings. These external catheter systems typically include a collection device, a collection canister, and a vacuum source. The collection device is configured to be positioned externally of the patient adjacent the urethra of the patient and to wick urine away from the patient. Various configurations of male and female collection devices are known and used for this purpose. The collection device is connected in fluid flow communication with the collection canister via a conduit such as flexible tubing. The interior of the collection canister is also in fluid flow communication with a vacuum source (e.g. a vacuum pump) such that a negative pressure created in the interior of the canister via the vacuum is effective to draw urine from the collection device through the conduit into the canister. Examples of external catheter systems are shown and described in WO 2024/112339, US 2024/0268986, US 2024/0268989, and US 2020/0315837. In many hospitals and other healthcare facilities, the collection canister is contained within a housing that is secured to the wall of the patient's room adjacent a vacuum source port and connected to the vacuum source port via flexible tubing.

The collected urine may be removed from the canister by various means depending on the overall configuration of external catheter system being used. In some instances, removal of the collected urine requires disassembling or disconnecting parts of the system to remove the canister, or changing the operating mode of the system to a discharge mode. The urine collected in the canister may be removed and disposed of on a set schedule by specifically assigned personnel, or by the patient's on-call nurse or other healthcare provider when they are not busy with other duties. Some systems are set up to run a cleaning solution through the canister as the urine is being discharged for disposal, while others may require disposal of the canister and replacement with a new canister.

From time to time there is a need for a nurse or other healthcare provider to capture a sample of urine from a patient for testing purposes. For those patients utilizing an external catheter system, the urine collected in the canister could potentially be used for such purposes, but depending on when the canister was last emptied, the collected urine may not be representative of a patient's current urine stream. In addition, removing a sample of urine from the canister may require the nurse or other healthcare provider to expend more time in disassembly, disconnecting, disposal and/or cleaning of the canister than they have at the time that a sample is needed.

### BRIEF SUMMARY OF THE INVENTION

The present invention is directed to an apparatus, kit, system and method for capturing a urine sample from a patient who is using an external catheter system, without removing urine from the collection canister of the external catheter system. The invention is fast and easy for nurses and other healthcare providers to use in capturing real-time urine samples and can be used for obtaining “clean catch” urine samples.

One aspect of the invention is directed to a method for collecting a urine sample from a patient. The method includes the following steps: (i) providing an external catheter system having a urine collection device, and a collection canister connected in fluid flow communication with a vacuum source; (ii) connecting the interior of a sample container in fluid flow communication with and between the urine collection device and the collection canister; (iii) permitting urine to be drawn from the urine collection device into the interior of the sample container; and (iv) disconnecting the interior of the sample container from fluid flow communication with and between the urine collection device and the collection canister. In some embodiments, the method includes after step (iv), the additional step of (v) connecting the urine collection device in fluid flow communication with the collection canister for normal operation of the external catheter system.

Another aspect of the invention is directed to an apparatus for obtaining a urine sample from the collection device of an external catheter system. The apparatus includes a lid configured to be removably secured to a sample collection container. The lid has a surface configured to cover an open top of the sample collection container, wherein the surface has an exterior face and an interior face. The lid also includes a connector having (i) a main body extending outward from the exterior face of the surface with an open end, (ii) a first arm extending outward from the main body with an open end, and (iii) a second arm extending outward from the main body with an open end. The connector and the surface together define a passageway for fluid flow communication between all of the open ends and through the surface. In some embodiments, the main body extends through the surface with the open end of the main body extending outward from the interior face. In some embodiments, the first and second arms are each configured to be releasably secured in fluid flow communication with first and second conduits respectively. In some embodiments, the first and second conduits each comprise flexible tubing. In some embodiments, the apparatus additionally comprises a first conduit secured to the open end of the first arm, and a second conduit releasably secured to the open end of the second arm. In some embodiments, the apparatus additionally includes a specimen or sample container, and the lid is removably secured to the container such that the open end of the main body extends into the interior of the container. In some embodiments, the apparatus additionally includes an external urine collection device and the first conduit has a first end releasably secured in fluid flow communication with the external urine collection device and a second end releasably secured in fluid flow communication with the open end of the first arm. In some embodiments, the apparatus additionally includes a collection canister in fluid flow communication with a vacuum source, and the second conduit has a first end releasably secured in fluid flow communication with the collection canister and a second end releasably secured in fluid flow communication with the open end of the second arm.

Another aspect of the invention is directed to an apparatus for obtaining a urine sample, the apparatus comprising a lid configured to be removably secured to a container, wherein the lid has a port extending through a surface of the lid, and wherein the lid comprises a connector, the connector having a first arm with a first open end and a second arm with a second open end, and wherein the connector defines a passageway for fluid flow communication between the first and second open ends and the port.

Another aspect of the invention is directed to a kit for retrofitting an external catheter system to enable a urine sample to be collected from the collection device of the external catheter system. The kit includes one of the apparatuses as described above. The kit may additionally include one or more of the following: flexible tubing, a connector plug configured to connect two pieces of flexible tubing together in fluid flow communication, a container configured such that the lid may be releasably secured to the container while collecting the sample, a closed top configured to be removably secured to the container to seal the container after the sample is collected, one or more urine collection devices, a collection canister, and/or a vacuum source.

Another aspect of the invention is directed to a system for collecting a urine sample of a test subject from the collection device of an external catheter. The system includes (i) an external urine collection device configured to be positioned adjacent a urethra of a test subject and to collect urine from the test subject; (ii) a storage canister configured to store urine collected by the external urine collection device, wherein the urine collection canister is connected in fluid flow communication with a vacuum source; and (iii) a sample collection apparatus configured to be temporarily secured in fluid flow communication between the external urine collection device and the urine collection canister to collect a urine sample from the urine collection device.

In some embodiments, the system additionally includes conduit configured to be connected in a normal external catheter operating configuration and in a sample collection configuration. In the normal operating configuration, the conduit extends from a first open end connected in fluid flow communication with the external urine collection device to a second open end connected in fluid flow communication with the storage canister. In the sample collection configuration, the conduit comprises (i) a first piece of conduit extending from a first open end connected in fluid flow communication with the external urine collection device to a second open end connected in fluid flow communication to the sample collection apparatus, and (ii) a second piece of conduit extending from a first open end connected in fluid flow communication with the sample collection apparatus to a second open end connected in fluid flow communication with the storage canister.

In some embodiments, the sample collection apparatus of the system includes a container with a lid removably secured to the container such that the lid surface covers the interior of the container. The lid includes a connector that has an open bottom end that extends through or adjacent to a port in the lid surface for access to the interior of the container. The connector also includes a first arm with an open end extending outward from the base, and a second arm with an open end extending outward from the base. The connector and the surface define a passageway for fluid flow communication between the open ends of the first and second arms and the interior of the container. In some embodiments, the first arm is configured to be releasably connected in fluid flow communication with the external urine collection device via conduit and the second arm is configured to be releasably connected in fluid flow communication with the storage canister via conduit. In some embodiments, when in the sample collection configuration, the first arm is connected in fluid flow communication with the first piece of conduit, and the second arm is connected in fluid flow communication with the second piece of conduit.

### BRIEF DESCRIPTION OF THE DRAWINGS

FIG. 1 depicts a urine sample collection apparatus in accordance with an embodiment of the present invention, the sample collection apparatus shown in an enlarged exploded view being connected to an external catheter system.

FIG. 2 is a perspective view of the top of the lid of the sample collection apparatus of FIG. 1.

FIG. 3 is a perspective view of the bottom of the lid of the sample collection apparatus of FIG. 1.

FIG. 4 is a sectional view of the lid of FIG. 2 taken along line 4-4 of FIG. 2 to show the passageway defined by the connector.

FIG. 5 is a top view of an alternative lid for a sample collection apparatus in accordance with the present invention.

FIG. 6 depicts attachment of the alternative lid of FIG. 5 to a sample container for sample collection.

FIG. 7 depicts connection of the ends of flexible tubing to the connector arms of the alternative lid of FIG. 5.

FIG. 8 depicts a top view of the alternative lid of FIG. 5 with tubing connected to the connector arms.

FIG. 9 depicts the alternative lid of FIG. 5 secured to a sample container, wherein tubing is connected to connector arms of the lid.

FIG. 10 depicts the alternative lid of FIG. 5 wherein the tubing is being removed from the connector arms of the lid.

FIG. 11 depicts securing a closed top to a sample container after collection of a urine sample.

FIG. 12 depicts the ends of the tubing of the sample collection apparatus depicted in FIG. 1 being secured together via a joinder plug for normal operation of the external catheter system.

### DETAILED DESCRIPTION OF PREFERRED EMBODIMENT

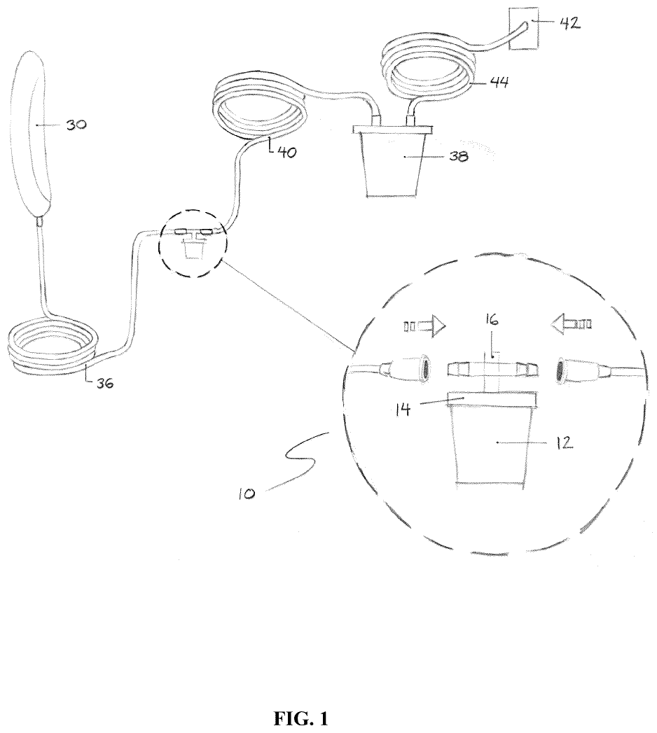

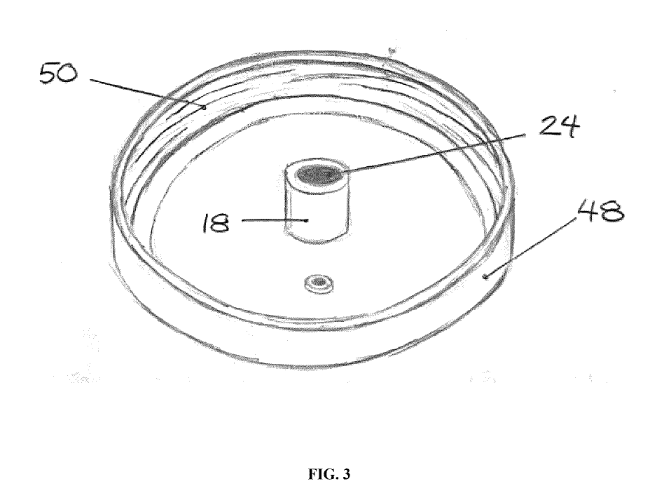

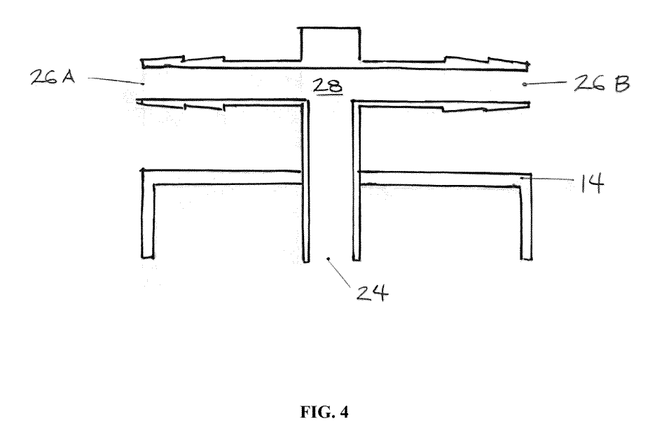

A urine sample collection apparatus in accordance with an embodiment of the present invention is generally designated by numeral 10 in FIG. 1. Sample collection apparatus 10 includes a sample collection cup 12 connected in-line with an external catheter system to obtain a urine sample from a patient who is using the external catheter system. Lid 14 secured over the open top of cup 12 includes a T-shaped hollow connector 16. As shown in FIGS. 1-4, connector 16 has a main body 18 extending through surface 20 of lid 14, and two arms 22A, 22B, extending outward from an upper portion of main body 18. Main body 18 extends from closed head end 23 through the lid surface 20 to an open tail end 24 (see FIG. 3). In this manner, the closed head end is positioned on the exterior of cup 12 above the lid surface and the open tail end 24 is positioned in the interior of the cup 12 below the lid surface. As shown in FIG. 4, connector 16 defines an internal passageway 28 for fluid flow between open end 26A of arm 22A, open end 26B of arm 22B, and open tail end 24 of main body 18.

Looking back to FIG. 1, open end 26A of arm 22A is connected to the external urine collection device 30 of an external catheter system via flexible tubing 36, and open end 26B of second arm 22B is connected to the collection canister 38 of an external catheter system via flexible tubing 40. Collection canister 38 is also connected to vacuum source 42 via flexible tubing 44. Flexible tubing 36, 40 and 44 enables fluid flow communication between collection device 30, sample collection cup 12, canister 38 and vacuum source 42. The vacuum provided by vacuum source 42 creates a negative pressure in sample collection cup 12 such that urine collected via collection device 30 is drawn through flexible tubing 36 into passageway 28 through open end 26A and into the interior of cup 12 through open tail end 24.

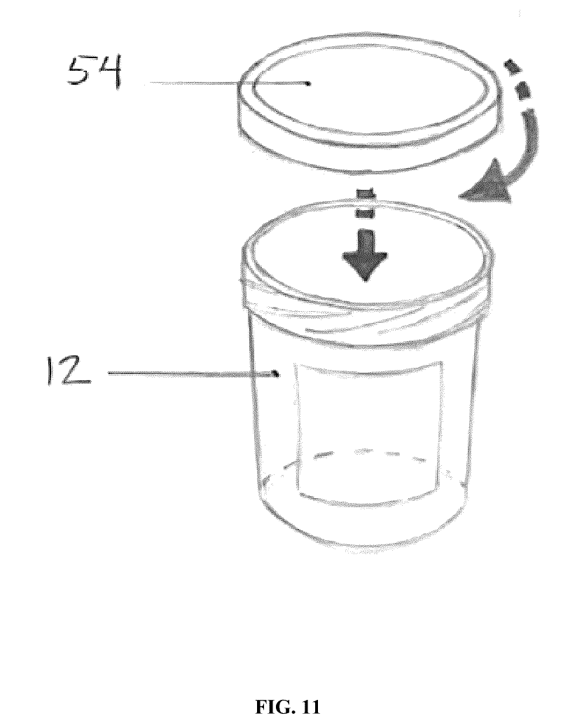

Urine collection device 30, collection canister 38, vacuum source 42, and flexible tubing 36, 40 and 44 collectively make up an external catheter system as is known in the art for use in collecting urine from a patient. When the sample collector apparatus of the present invention is not in use to collect a urine sample from the patient, the ends of flexible tubing 36, 40 are disconnected from arms 22A, 22B of connector 16 respectively and are connected together via tubular joinder plug 46 (see FIG. 11) for normal operation of the external catheter system. When a urine sample is needed from the patient, the ends of flexible tubing 36, 40 are disconnected from one another and connected to open ends 26A, 26B of arms 22A, 22B respectively to collect the urine sample collected by the collection device when the patient next voids his or her bladder.

While the external catheter system depicted in FIG. 1 includes a specific configuration of a female urine collection device, it should be understood that any type (male or female) and configuration of external urine collection device is considered suitable for purposes of this invention. In addition, various different configurations of collection canisters and vacuum sources are known and could be used in conjunction with the sample collection apparatus. In FIG. 1, the vacuum source is depicted as a vacuum port as is typically provided on the wall of a hospital or other healthcare facility. The vacuum port typically would be connected via conduit to one or more vacuum pumps located on the facility premises. Of course, the vacuum could alternatively be provided in another manner such as by a pump in the room of the patient or by a pump integrated with the collection canister.

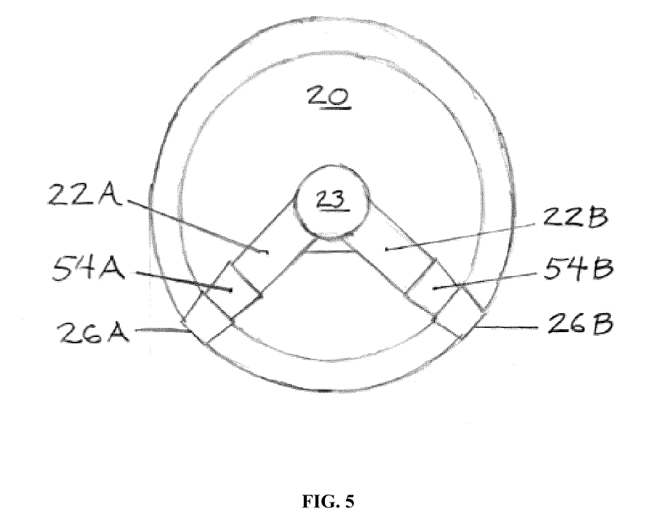

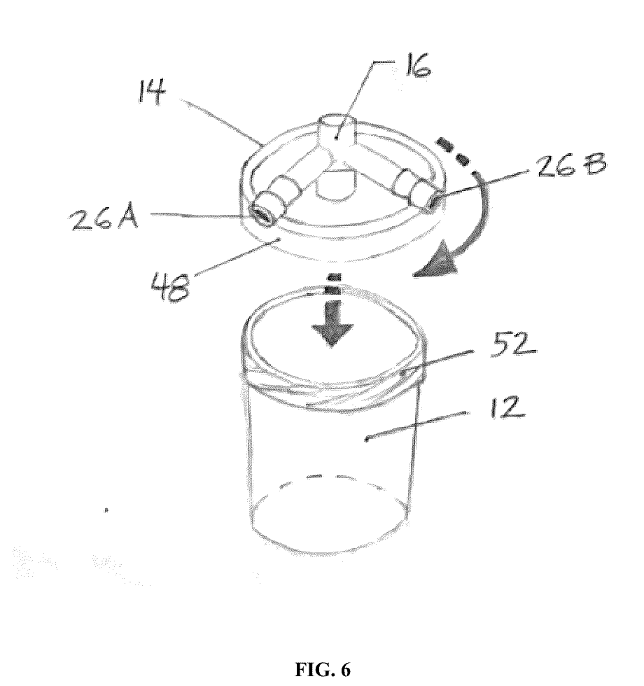

Looking to FIGS. 3 and 5, lid 14 has peripheral skirt 48 extending downward from the outer edge of top surface 20. Skirt 48 is configured to fit around and engage the upper portion of the outer sidewall of cup 12. The inner surface of peripheral skirt 48 includes threading 50 that cooperates with corresponding threading 52 (FIG. 6) on the upper outer surface of cup 12 in order for lid 14 to be screwed onto the top of cup 12. It should be understood that other means for temporarily securing lid 14 to cup 12 may be utilized without departing from the invention, such as via a press fit, an interference fit or latches. In some embodiments, the lid 14 and cup 12 are connected in a manner to create an air tight seal such that the desired negative pressure within the interior of the cup will draw urine into the cup. One or more rubber gaskets (not shown) may be presented between the lid 14 and cup 12 to assist in sealing the connection.

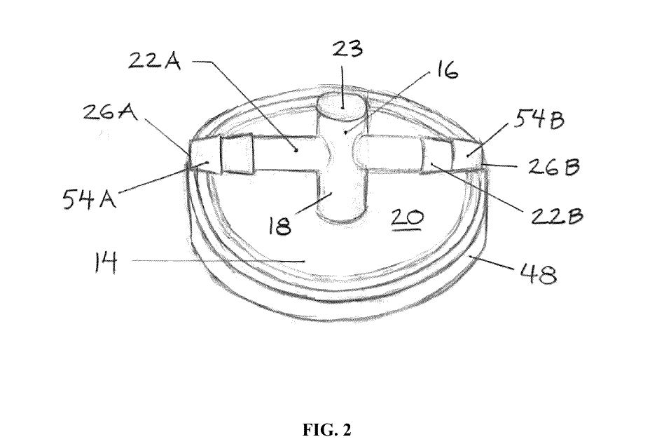

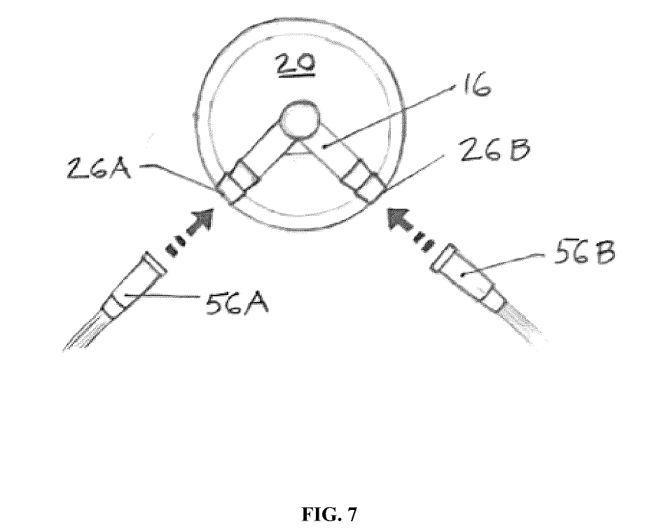

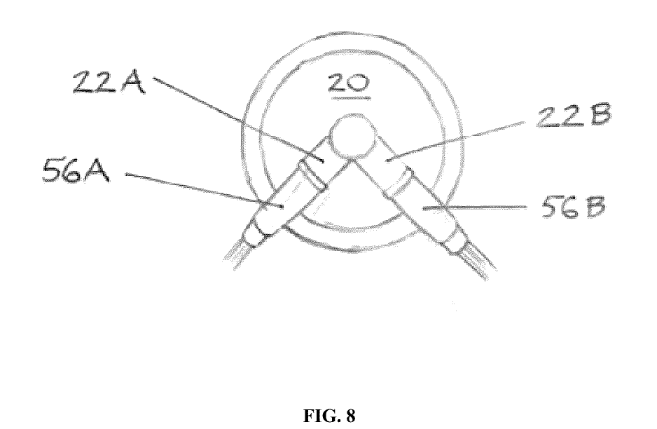

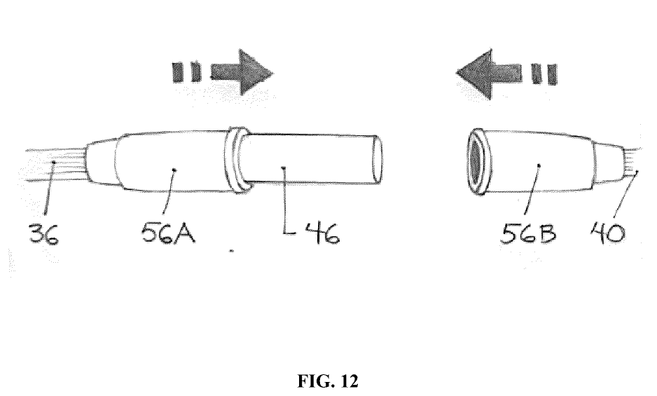

Looking to FIGS. 2, 4, 7 and 8, arms 22A, 22B each include a plurality of conical ridges 54A, 54B configured to assist in connecting the flexible hoses 36, 40 in a sealed engagement with open ends 26A, 26B respectively. Looking also to FIG. 12, flexible hoses 36, 40 include rigid flared ends 56A, 56B to facilitate connecting the tubing in fluid flow communication with passageway 28 of apparatus 10 for sample collection and with joinder plug 46 for normal operation of the external catheter system.

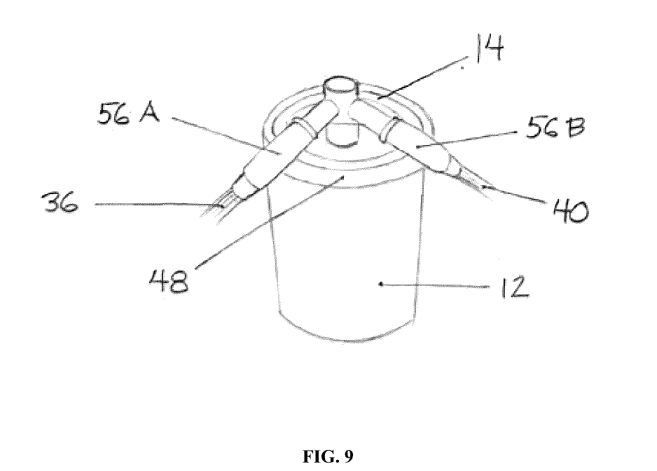

Looking to FIG. 2, arms 22A, 22B extend outwardly from opposite sides of main body 18 such that the arms extend outwardly approximately 180 degrees apart from one another. In an alternative embodiment shown in FIGS. 5-10, arms 22A, 22B are instead positioned approximately 90 degrees apart. The positioning of arms 22A, 22B relative to one another and to main body 18 may be at any angle and preferably will accommodate ease of use in positioning the sample cup 12 relative to external collection device 34 and canister 38. In addition, while the arms 22A, 22B shown in the figures extend outward from main body along the same vertical plane, it should be understood that arm 22A could extend outward from body 18 at a position that is higher or lower than arm 22B. Furthermore, while arms 22A, 22B extend outwardly essentially parallel the surface 20 of lid and perpendicular the longitudinal axis of main body 18, it should be understood that arms and main body may be positioned at various angles relative to one another and to the surface 20 of lid 14 without departing from the present invention.

In the embodiments shown in the figures, connector 16 is integrally formed with the remainder of the lid 14 such that passageway 28 extends through main body 18 to open tail end 24 within the interior of the container. It should be understood that connector 16 or parts thereof may be formed as a separate component and connected in fluid flow communication with the interior of the container. For example, open tail end 24 could be secured to the lid surface 20 adjacent an opening or port in the lid surface to provide fluid flow access to the interior of the container.

The sample collection device may be used to collect a urine sample from a patient that is connected to an external catheter system at various times as needed without the need for removing collected urine from the collection canister. It is envisioned that a urine sample may be taken from a patient after the patient is initially connected to a clean collection device for the day, and/or at various other times as needed on a real time basis. When a urine sample is needed, the sample collection apparatus is installed in-line with the remaining components of the external catheter system. When the sample collection is completed, the sample collection apparatus is disconnected, the disconnected tubing is connected together, and the external catheter system returns to normal operation.

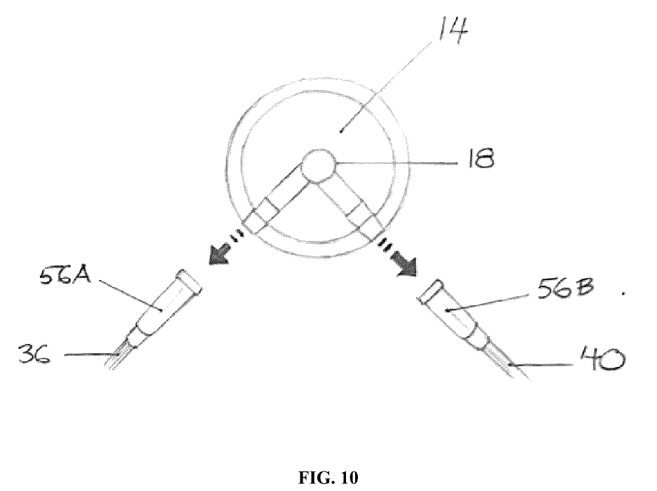

Looking to FIG. 1, in operation the collection device 30 is positioned externally of a patient and secured in place adjacent the urethra of the patient. When a urine sample is desired to be taken, lid 14 is secured to a specimen container 12 (FIG. 6). Rigid end 56A of tubing 36 is connected to open end 26A of arm 22A, and rigid end 56B of tubing 40 is connected to open end 26B of arm 22B (FIGS. 1 and 7-9). When the patient voids his or her bladder, the urine is collected by the collection device and is drawn through tubing 36 and passageway 28 into the interior of container 12 by the negative pressure created in the interior of the container via the vacuum source 42. As shown in FIG. 10, once the urine sample is collected in the container 12, rigid end 56A of tubing 36 is disconnected from open end 26A of arm 22A, and rigid end 56B of tubing 40 is disconnected from open end 26B of arm 22B. Lid 14 is then removed and, as shown in FIG. 11, a closed cover cap 54 is secured over the open top of container 12 to close the container and protect against spillage of the collected urine sample. As shown in FIG. 12, a tubular joinder plug 46 may then be inserted into rigid ends 56A, 56B to connect tubing 36 and 40 together in fluid flow communication for normal operation of the external catheter system.

In some embodiments, it is anticipated that a kit for sample collection may be provided wherein the kit includes lid 14 with connector 16 configured to fit on a standard urine sample cup. The kit may additionally include additional kit components selected from the group consisting of one or more of the following: sample cup 12; tubing 36, 40; joinder plug 46; top cover 54; and combinations thereof. In addition and/or alternatively, the lid 14 may be provided in a kit with one or more components of an external catheter system selected from the group consisting of one or more of the following: urine collection device 30, collection canister 38, tubing 44, vacuum source 42 (e.g. pump) and combinations thereof.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. An apparatus for obtaining a urine sample from the collection device of an external catheter, the apparatus comprising:
a lid configured to be removably secured to a sample collection container, the lid comprising:
a surface configured to cover an open top of the sample collection container, wherein the surface comprises an exterior face and an interior face; and
a connector comprising (i) a main body extending outward from the exterior face of the surface, wherein the main body has an open end, (ii) a first arm extending outward from the main body, wherein the first arm has an open end, and (iii) a second arm extending outward from the main body, wherein the second arm has an open end; and
wherein the connector and surface together define a passageway for fluid flow communication between the open ends and through the surface.

2. The apparatus of claim 1, wherein the main body extends through the surface with the open end of the main body extending outward from the interior face.

3. The apparatus of claim 1, wherein the first and second arms are each configured to be releasably secured in fluid flow communication with first and second conduits respectively.

4. The apparatus of claim 3, wherein the first and second conduits each comprise flexible tubing.

5. The apparatus of claim 3, wherein the apparatus additionally comprises a first conduit releasably secured to the open end of the first arm, and a second conduit releasably secured to the open end of the second arm.

6. The apparatus of claim 2, wherein the apparatus additionally comprises a container, and wherein the lid is removably secured to the container such that the open end of the main body extends into an interior of the container.

7. The apparatus of claim 5, wherein the apparatus additionally comprises an external urine collection device and wherein the first conduit comprises a first end releasably secured in fluid flow communication with the external urine collection device and a second end releasably secured in fluid flow communication with the open end of the first arm.

8. The apparatus of claim 5, wherein the apparatus additionally comprises a collection canister in fluid flow communication with a vacuum source, and wherein the second conduit comprises a first end releasably secured in fluid flow communication with the collection canister and a second end releasably secured in fluid flow communication with the open end of the second arm.

9. An apparatus for collecting a urine sample, the apparatus comprising a lid configured to be removably secured to a container, wherein the lid has a port extending through a surface of the lid, and wherein the lid comprises a connector, the connector having a first arm with a first open end and a second arm with a second open end, and wherein the connector defines a passageway for fluid flow communication between the first and second open ends and the port.

10. A kit for retrofitting an external catheter system to enable a urine sample to be collected from the collection device of the external catheter system, said kit comprising:
a. a lid configured to be removably secured to a container, wherein the lid has a port extending through a surface of the lid, and wherein the lid comprises a connector, the connector having a first arm with a first open end and a second arm with a second open end, and wherein the connector defines a passageway for fluid flow communication between the first and second open ends and the port;
b. flexible tubing; and
c. a connector plug configured to connect two pieces of flexible tubing together in fluid flow communication.

11. The kit of claim 10 additionally comprising one or more urine collection devices of an external catheter system.

12. The kit of claim 10 additionally comprising a container configured such that the lid may be releasably secured to the container while collecting the sample.

13. The kit of claim 11 additionally comprising a closed top configured to be removably secured to the container to seal the container after the sample is collected.

14. A system for collecting a urine sample of a test subject from the collection device of an external catheter, the system comprising:
an external urine collection device configured to be positioned adjacent a urethra of a test subject and to collect urine from the test subject;
a storage canister configured to store urine collected by the external urine collection device, the urine collection canister in fluid flow communication with a vacuum source; and
a sample collection apparatus configured to be temporarily secured in fluid flow communication between the external urine collection device and the urine collection canister to collect a urine sample from the urine collection device.

15. The system of claim 13, wherein said system additionally comprises conduit configured to be connected in a normal operating configuration and in a sample collection configuration, wherein in the normal operating configuration the conduit extends from a first open end connected in fluid flow communication with the external urine collection device to a second open end connected in fluid flow communication with the storage canister, and wherein in the sample collection configuration the conduit comprises a first piece of conduit extending from a first open end connected in fluid flow communication with the external urine collection device to a second open end connected in fluid flow communication with the sample collection apparatus, and a second piece of conduit extending from a first open end connected in fluid flow communication with the sample collection apparatus to a second open end connected in fluid flow communication with the storage canister.

16. The system of claim 15, wherein the sample collection apparatus comprises a container with a lid removably secured to the container such that the lid surface covers the interior of the container, the lid comprises a connector having a base that extends through or adjacent a port in the lid surface for access to the interior of the container, a first arm with an open end extending outward from the base, and a second arm with an open end extending outward from the base, wherein the connector and surface define a passageway for fluid flow communication between the open ends of the first and second arms and the interior of the container.

17. The system of claim 16, wherein the first arm is configured to be releasably connected in fluid flow communication with the external urine collection device via conduit and the second arm is configured to be releasably connected in fluid flow communication with the storage canister via conduit.

18. The system of claim 17, wherein in the sample collection configuration the first arm is connected in fluid flow communication with the first piece of conduit, and the second arm is connected in fluid flow communication with the second piece of conduit.

19. A method for collecting a urine sample from a patient, wherein the method comprises:
(i) providing an external catheter system comprising a urine collection device, and a collection canister connected in fluid flow communication with a vacuum source;
(ii) connecting an interior of a sample container in fluid flow communication with and between the urine collection device and the collection canister;
(iii) permitting urine to be drawn from the urine collection device into the interior of the sample container; and
(iv) disconnecting the interior of the sample container from fluid flow communication with and between the urine collection device and the collection canister.

20. The method of claim 19 additionally comprising after the disconnecting step the following step: (v) connecting the urine collection device in fluid flow communication with the collection canister for normal operation of the external catheter system.
